(19) United States
(12) Patent Application Publication
(10) Pub. No.: US 2004/0089292 A1
(43) Pub. Date: May 13, 2004

(54) Dispensing apparatus

(76) Inventors: Hilde Rachel Maria Pollet, Cambridge (BE); Sally Beken, Hampshire (GB)

Correspondence Address: MORRIS, MANNING & MARTIN LLP, 6000 FAIRVIEW ROAD, SUITE 1125, CHARLOTTE, NC, 28210, US

(21) Appl. No.: 10/332,154

(22) PCT Filed: Jun. 28, 2001
(86) PCT No.: PCT/GB01/02897

(30) Foreign Application Priority Data
Jul. 6, 2000 (GB) 0016670.2
Aug. 21, 2000 (GB) 0020600.3

(51) Int. Cl. A61M 11/00

(52) U.S. Cl. 128/200.23; 128/200.14; 604/58; 222/402.1

(57) ABSTRACT

The invention relates to a dispensing apparatus for pharmaceutical porducts and preparations having improved tactile and hygiene features. where the dispensing apparatus comprises an outlet through which, in use, product is dispensed from a container of stored product located there within. At least a portion of an exterior surface of the apparatus being formed from a thermoplastic elastomer.

This invention relates to dispensing apparatus. In particular, the invention relates to dispensing apparatus for pharmaceutical products and preparations having improved tactile and hygiene features.

Many types of dispensing apparatus are known for use in dispensing pharmaceutical products and preparations. These include oral actuators, nasal actuators, spacers, ophthalmic dispensers, dermal applicators (transdermal or hypodermic), needleless injectors, aural dispensers, vaginal dispensers and the like.

Oral actuators typically comprise a container in which the medicament is stored. Such containers include a dispensing container, in which the medicament is stored as a liquid medium. The dispensing container may be ‘pressurised’ wherein the medicament is dispensed using a volatile propellant or ‘unpressurised’ wherein the medicament is dispensed using a pump or other non-liquified gas system. Alternatively, the medicament can be dispensed from frangibly sealed containers, in which the medicament is stored as a dry powder and dispensed using an air stream. Variants of such oral actuators include metered dose inhalers, pressurised metered dose inhalers, dry powder inhalers, breath actuated inhalers and breath co-ordinated inhalers.

Nasal actuators typically comprise a vial or other container for storage of the medicament. Dispensation of the medicament via an outlet nozzle is achieved by use of a volatile propellant, driven by an air stream or by means of a pressure being applied to the liquid medicament via a drive means. Alternatively, dispensation may be gravity driven, as in the case of ‘droppers’ for nose drop preparations.

Spacers are used in conjunction with another dispensing apparatus to provide a chamber or other generally enclosed space in which the velocity of dispensed medicament particles may be slowed before they are administered to a user, either orally or nasally.

Ophthalmic dispensers include ‘droppers’ for eye drop preparations, eye-baths for bathing of the eye with a medicament ‘wash’ and dispensing containers (either pressurised or unpressurised) for dispensing medicaments as low velocity mists towards the eye.

Dermal applicators include hypodermic syringes, catheters and other such devices which physically penetrate the skin boundary layer.

Needleless injectors, unlike hypodermic syringes, are used for dispensing medicaments subcutaneously but without any portion of the injector penetrating the skin boundary layer. Typically such injectors work by driving the medicament at high velocities across the skin boundary using a compressed medium such as compressed air to provide the driving force.

Aural dispensers include ‘droppers’ for ear drop preparations and other such applicators for applying liquids, creams or the like into the ear.

Vaginal dispensers include devices for applying liquids, creams and like preparations into the vagina.

A common feature of all of the above described apparatus is that an outlet is provided through which the medicament is dispensed. The outlet may be a mouthpiece in the case of, for example, oral actuators and spacers or a generally conically-shaped tip in the case of, for example, nasal, aural, dermal or vaginal actuators. The outlet is typically contacted by the user during use. Therefore, it is important that the surfaces of the outlet are kept free from dirt, dust and other contaminants. In addition, it is advantageous to provide a means for sealing the outlet of the apparatus to prevent the ingress of dirt, dust or other contaminants into the body of the apparatus.

One problem with such dispensing apparatus is that the material of the apparatus can cause discomfort where it comes into contact with the user during dispensation. For example, known polymeric plastics such as polypropylene and high density polyethylene can feel ‘cold’ to the touch and may become abraided during use, forming sharpened edges or corners which may cause pain or injury to a user. For example, the mouthpiece or sealing cap of an oral actuator may scratch or cut the tongue, lips or face of a user if any sharpened edges or corners are present. Likewise, the tip of a nasal actuator may cut or scratch the nasal lining of a user if it becomes roughened.

Another problem with such dispensing apparatus is that they can be difficult to grasp and handle especially by the young or infirm. Typical polymeric plastics used for the housings of such dispensing apparatus tend to be reasonably slippery especially when wet. In addition the apparatus can be difficult to grasp and operate especially one-handed.

According to the present invention, there is disclosed a hand held dispensing apparatus comprising an outlet through which, in use, product is dispensed from a container of stored product locatable within, or communicating with, the apparatus wherein at least a portion of an exterior surface of the apparatus is formed from a thermoplastic elastomer.

Embodiments of the present invention will now be described, by way of example only, with reference to the accompanying drawings, in which:

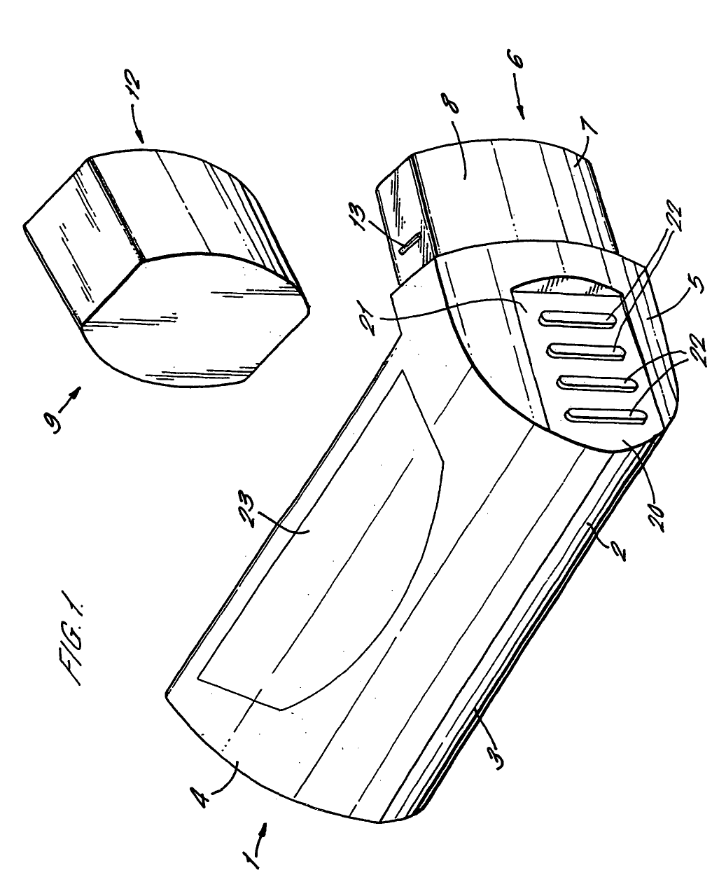

FIG. 1 is perspective view of a first embodiment of dispensing apparatus according to the present invention;

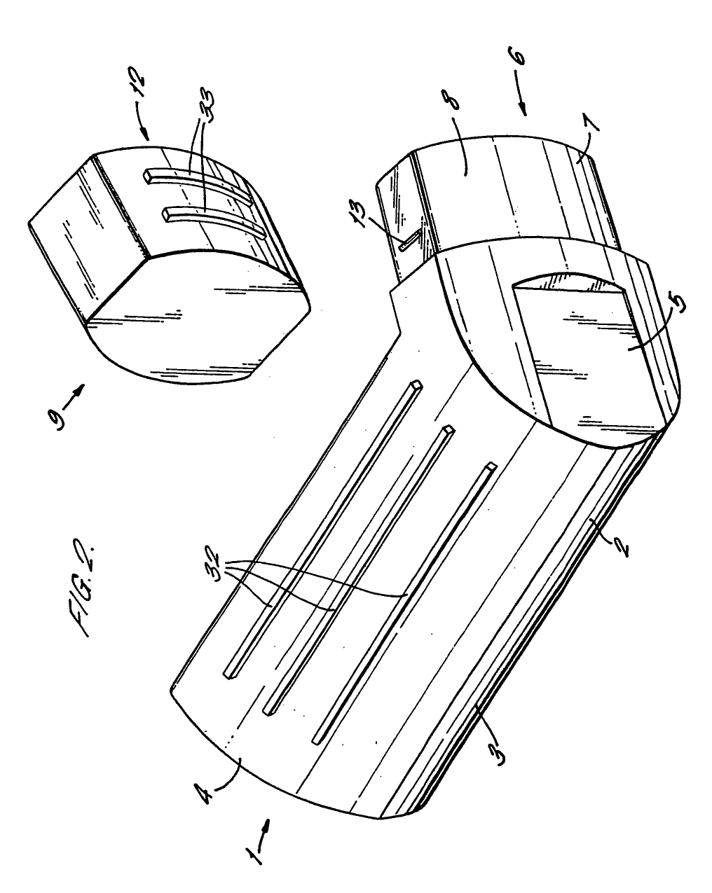

FIG. 2 is perspective view of a second embodiment of dispensing apparatus according to the present invention;

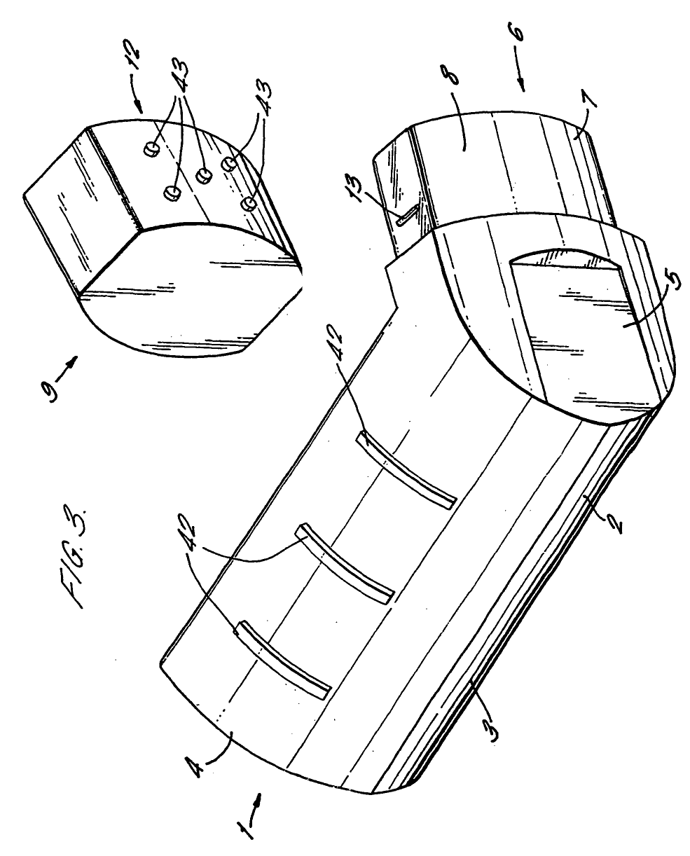

FIG. 3 is perspective view of a third embodiment of dispensing apparatus according to the present invention;

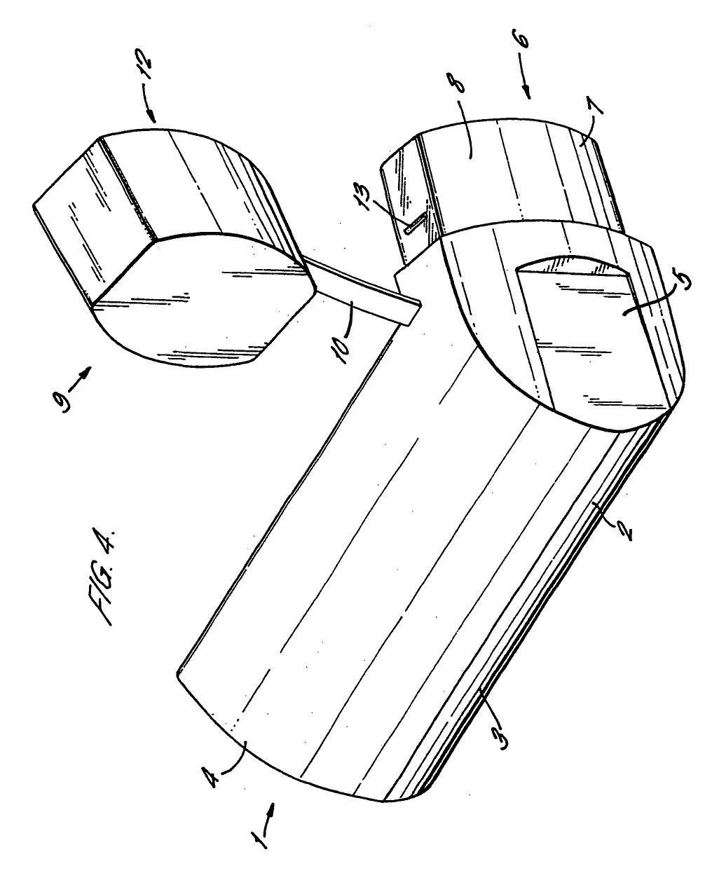

FIG. 4 is a perspective view of a fourth embodiment of apparatus according to the present invention having an integrally formed strap attached to a side wall and with the cap disengaged from the mouthpiece.

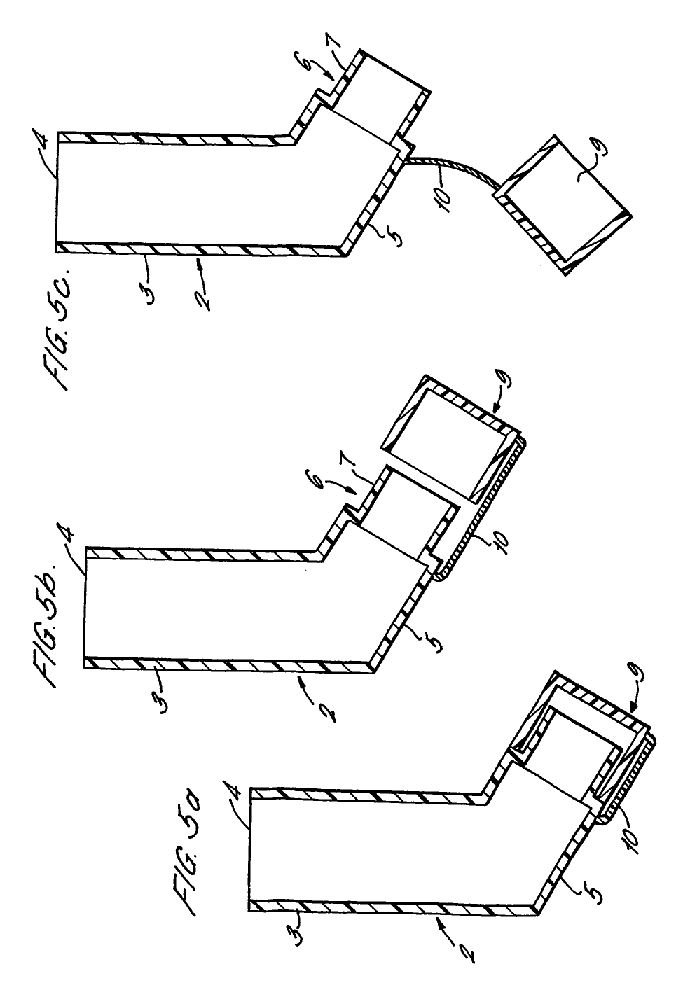

FIG. 5a is a cross-sectional schematic view of a fifth embodiment of apparatus according to the present invention having an integrally formed strap attached to an end wall and the cap being in the engaged position on the mouthpiece;

FIG. 5b is a cross-sectional schematic view of the apparatus of FIG. 5a showing the cap in co-axial alignment with the mouthpiece immediately prior to movement of the cap into the engaged position on the mouthpiece;

FIG. 5c is a cross-sectional schematic view of the apparatus of FIG. 5a showing the cap in an unengaged position, for example, during actuation of the apparatus;

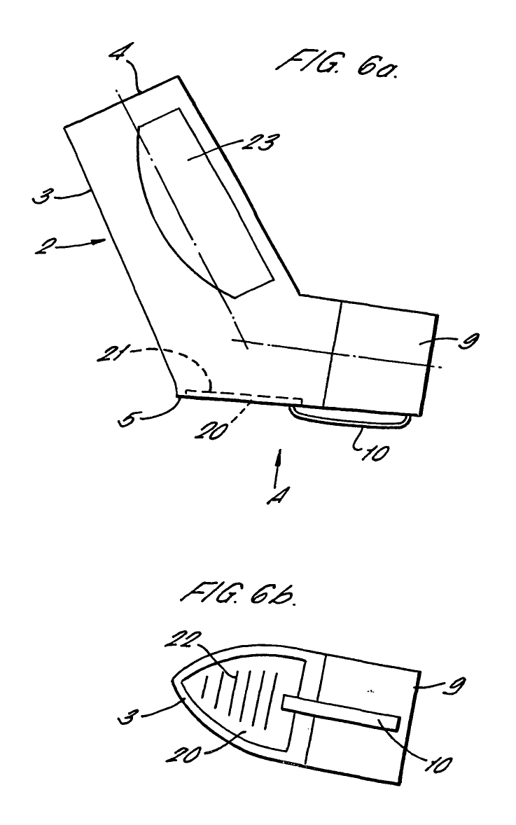

FIG. 6a is side view of a sixth embodiment of dispensing apparatus according to the present invention;

FIG. 6b is an end view of the apparatus of FIG. 6a;

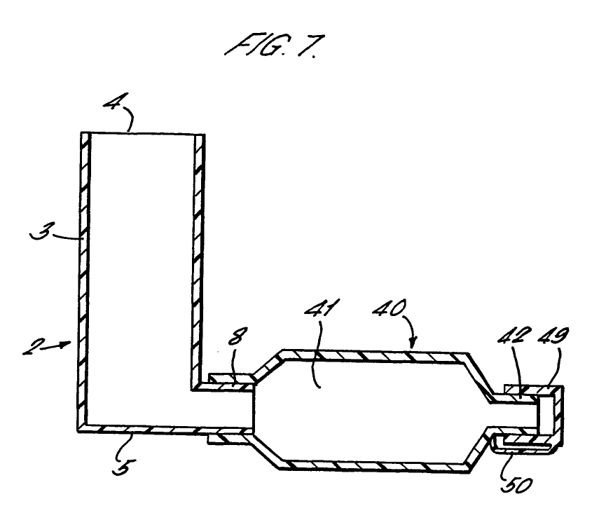

FIG. 7 is a sectional view of a seventh embodiment of dispensing apparatus according to the present invention;

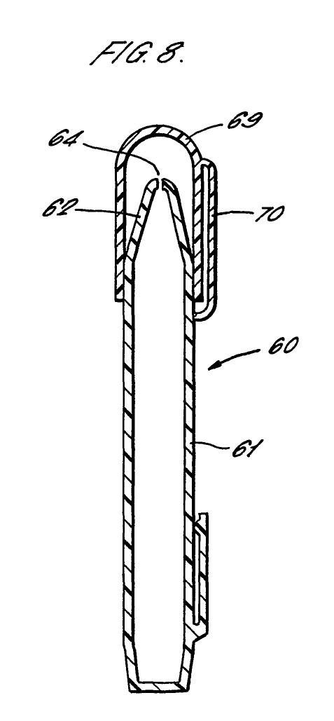

FIG. 8 is a sectional view of an eighth embodiment of dispensing apparatus according to the present invention; and

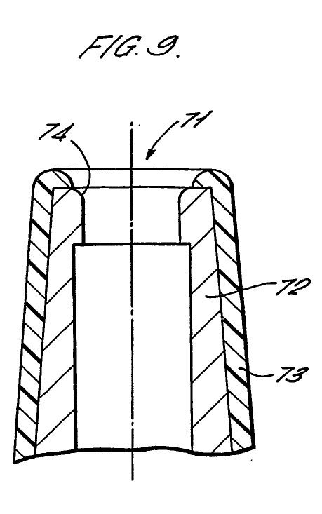

FIG. 9 is an enlarged sectional view of the outlet of the apparatus of FIG. 8.

FIGS. 1 through 6b show six embodiments of the present invention. The embodiments have in common an apparatus 1 comprising a housing 2 consisting of a tubular body 3 having a tubular side wall and an open end 4. The body 3 is closed at its opposite end by an end wall 5 and a tubular mouthpiece 6 projects laterally of the body at a location immediately adjacent the end wall 5. The mouthpiece 6 has a tubular lip portion 7 having an external surface 8 which in use is presented to the lips of a user wishing to inhale orally via the mouthpiece an aerosol spray generated from a pressurised dispensing container (not shown) normally received within the body 3.

The apparatus 1 further comprises a cap 9. The cap 9 is a sliding fit onto the lip portion 7 such that an internal surface 12 of the cap totally overlays the external surface 8 of the lip portion when the cap is moved into an engaged position in which it is engaged with the mouthpiece 6.

The lip portion 7 and the cap 9 are provided with co-operating snap fit connectors which include a detent 13 projecting from the lip portion in co-operating relationship with a groove (not shown) formed in the internal surface 12 of the cap 9.

In known apparatus the housing 2 and cap 9 are typically formed from a plastics material such as polypropylene or high density polyethylene (HDPE).

According to the present invention the apparatus is formed as a co-moulding of HDPE, polypropylene or similar together with a thermoplastic elastomer to form one or more surface features on the apparatus 1 to improve the apparatus' hygiene and/or tactile features.

In a first step of the co-moulding the housing 2 is moulded from polypropylene, HDPE or a similar material. In a second step the thermoplastic elastomer components of the apparatus are moulded intimately with the housing 2. The thermoplastic elastomer components are retained on the housing 2 by a mixture of physical and chemical bonding. In addition, and if required, additional fixing means may be used to provide a superior bond, e.g. welding, mechanical fasteners, glue etc.

Preferably both steps of the co-moulding process are carried out in the same mould tool. Alternatively, the moulded housing 2 may be moved to a different mould tool for the second step. The two mould steps could be reversed in order. The thermoplastic elastomer used can be any of Santoprene, Pebax, Vitaprene, Hytrel or the like. Various thermoplastic elastomer components may be advantageously moulded onto the housing 2 as described below.

In the first embodiment, shown in FIG. 1, a pad 20 of a thermoplastic elastomer is formed on end wall 5. Preferably, the pad 20 is provided with ridges 22 or other surface protrusions or indentations. These serve to improve the grip of the user's finger or thumb when holding the apparatus 1. The pad 20 is preferably “keyed” into a recessed “key-way” 21 in the end wall 5 of the tubular body 3. The “key” and “key-way” arrangement provides an improved connection between the thermoplastic elastomer and the polypropylene or HDPE material. Alternatively, the pad 20 may be simply bonded to the surface of the end wall 5 without the use of a “key-way”. The housing 2 may be provided with a re-entrant feature through which the pad 20 is moulded to provided for improved attachment of the pad 20 to the housing 2.

One or more portions 23 of the tubular body 3 are provided with a portion of thermoplastic elastomer. The thermoplastic elastomer portions 23 provide for an improved grip on the sides of the tubular body 3. The portions 23 may be bonded on the surface of the tubular body 3 or “keyed” into recesses formed in the surface. Writing and/or pictures may be formed in the portions 23 either as “cut-out” areas or as indentations or protrusions. In this way information such as dosage instructions, product warnings and brand logos may be provided on the housing 2. Raised writing or braille notation may be provided for the visually impaired. Advantageously, the writing and/or pictures, being an intimate part of the apparatus 1, are resistant to being removed or obliterated over time. This is especially important for dosage instructions and warning notices which have previously tended to be provided on sticky labels or similar, which may easily be removed.

In the second embodiment, shown in FIG. 2, a series of longitudinally orientated ridges 32 are formed on the sides of tubular body 3. The ridges are formed from a co-moulded thermoplastic elastomer of the type, and in the manner, described above. Ridges 33 may also be provided on cap 9. Preferably the ridges 33 are orientated laterally so as to improve the finger-grip of a user pulling or pushing on the cap 9. The ridges 32, 33 may be “keyed” into the surface or bonded onto the surface.

The third embodiment comprises a similar arrangement, shown in FIG. 3, except that the ridges 42 are diagonally orientated and that raised dots 43 are provided on the cap 9.

FIG. 4 shows a fourth embodiment of the present invention, whilst FIGS. 5a to 5c show a fifth embodiment of the present invention.

Referring to FIG. 4, the cap 9 is connected to the body 3 by a strap 10 formed of a thermoplastic elastomer material such as Santoprene (RTM), Pebax (RMT), Vitaprene (RTM), Hytrel (RTM) or the like. The strap 10 is joined to the cap 9 and housing 2 by mechanical fasteners, glue, heated or ultrasonic welds, or a combination of these means.

In the fourth embodiment, shown in FIG. 4, the cap 9 is attached to a side of the tubular body 3. In the fifth embodiment, shown in FIGS. 5a to 5c, the strap 10 is attached to the end wall 5. In other respects the fourth and fifth embodiments are identical. As such, the engagement and disengagement of the cap will now be described with reference to the fifth embodiment only.

Advantageously, the inherent elasticity of the thermoplastic elastomer material allows the strap 10 to stretch and extend in length when the cap 9 is pulled in a direction away from the tubular body 3.

The cap 9 is a sliding fit onto the lip portion 7 such that an internal surface 12 of the cap totally overlays the external surface 8 of the lip portion when the cap is moved into an engaged position as shown in FIG. 5a in which it is engaged with the mouthpiece 6.

The lip portion 7 and the cap 9 are provided with co-operating snap-fit connectors which include a detent 13 projecting from the lip portion in co-operating relationship with a groove (not shown) formed in the internal surface 12 of the cap 9.

As shown in FIG. 5c, the cap is movable when disengaged from the mouthpiece 8 into a position in which it lies at a location which is offset from the axial extent of the body 3 and from the axial extent of the mouthpiece 6 by a distance determined by the length of the strap 10. Due to the relative flexibility of the strap 10 the cap tends to ‘fall away’ from the mouthpiece 6 when it is disengaged which greatly improves access to the mouthpiece 6.

In this position, the user is able to grip the housing without interference from the presence of the cap 9 and strap 10, the user typically resting a thumb against the end wall 5 and an index finger around the barrel shaped body 3.

In order to move the cap 9 from this position of FIG. 5c into the engaged position of FIG. 5a it is necessary to move the cap 9 away from the body 3 into co-axial alignment with the mouthpiece 6 at a position in which the cap 9 extends beyond the axial extent of the lip portion 7, as shown in FIG. 5b. During this movement the strap 10 is stretched. The maximum extended length of the strap 10 must correspond at least to this configuration. Movement of the cap into the engaged position then proceeds by pushing the cap 9 towards the body 3 in sliding relationship relative to the lip portion 7 until the detent 13 effects a snap fit connection and the cap rests in the fully engaged position shown in FIG. 5a in which the entire external surface 8 of the lip portion 7 is overlaid.

In the engaged position of FIG. 5a it is seen that the strap 10 has elastically recovered to its original, unstretched length, such that the strap 10 is held close to the body 3 and does not extend away from the body 3 in a loop formation. As a result the risk of catching the strap 10 on an object and accidentally dislodging the cap 10 is greatly reduced. Also the overall dimensions of the apparatus in the storage condition are reduced.

Cap 9 may be formed from a plastics material such as polypropylene. Advantageously, according to a further aspect of the present invention, the cap 9 may also be formed from a thermoplastic elastomer. The cap 9 and strap 10 may then be formed as a unitary body by means of a moulding process. The inherent flexibility of the thermoplastic elastomer allows the cap 9 to be distorted or crushed and yet return to its original shape. This is advantageous in that it both simplifies removal and engagement of the cap 9 with the mouthpiece 8 as the cap 9 can distort somewhat but also in that the cap does not risk scratching the face of the user during use.

FIGS. 6a and 6b show a sixth embodiment of the present invention. Similar components to those shown in the previous embodiments have been referenced with the same reference numerals and will not be described here in any more detail. Strap 10 is connected to the cap 9 as in the fifth embodiment. At the other end of the strap 9 is formed a pad 20 of a thermoplastic elastomer material, such as Santoprene Pebax, Vitaprene, Hytrel or the like. The pad 20 may be connected to the strap 10 by any of the methods described above in connection with the previous embodiments. However, preferably the strap 10 and pad 20 are formed as a unitary body by a moulding process. In the case where the strap 10, cap 9 and pad 20 are all formed of a thermoplastic elastomer, preferably they are formed as a single unitary body by means of a moulding process.

The pad 20 serves as a “key” which is received in a recessed “key-way” 21 in the end wall 5 of the tubular body 3. The pad 20 is fixedly attached to the recessed “key-way” 21 by any of the methods described above in the first embodiment, e.g. welding, mechanical fasteners, glue etc. The “key” and “key-way” arrangement provides an improved connection between the thermoplastic elastomer and the polypropylene materials. Alternatively the pad 20 may be simply bonded to the surface of the end wall 5 without the use of a key-way.

Advantageously, where the cap 9, strap 10 and pad 20 are all formed from a thermoplastic elastomer the apparatus may be formed as a two-step moulding. In the first step the housing 2 is moulded from a polypropylene or similar material and in the second step the cap 9, strap 10 and pad 20 are moulded from a thermoplastic elastomer directly into position on the housing 2, with the pad 20 being retained on the housing 2 by a mixture of physical and chemical bonding. Preferably both steps of the moulding process are carried out in the same mould tool. Alternatively, the moulded housing 2 may be moved to a different mould tool for the second step. The two mould steps could be reversed in order.

Optionally the housing 2 is provided with a re-entrant feature through which the pad 20 is moulded to provided for improved attachment of the pad 20 to the housing 2.

Preferably the pad 20 is provided with ridges 22 or other surface protrusions or indentations. These serve to improve the grip of the user's finger or thumb when holding the apparatus.

FIG. 7 shows a seventh embodiment of dispensing apparatus in the form of a spacer 40 attached to an oral actuator of the type shown in FIGS. 1 to 6b.

The spacer 40 attaches to the mouthpiece 8 of the oral actuator and provides a chamber 41 in which the dispensed medicament particles slow before inhalation. A mouthpiece 42 generally opposite the mouthpiece 8 of the oral actuator is provided with a cap 49 which may be attached to the spacer body by means of a strap 50. The structure, materials and operation of the strap and cap arrangement are the same as described above. Of course, the strap and cap arrangement of the present invention may be applied to other types of spacer or where the spacer is attached to other types of actuator.

FIG. 8 shows an eight embodiment of the present invention comprising a nasal actuator 60 having a body 61, tip 62 and cap 69. The tip 62 defines an outlet 64 through which medicament is dispensed. The nasal actuator 60 is provided with a cap 69 which may be attached to the spacer body by means of a strap 70. The structure, materials and operation of the strap and cap arrangement are the same as described above.

The spacer 40 and nasal actuator 60 may comprise any of the thermoplastic elastomer components described with reference to the oral actuators of FIGS. 1 to 6b. For example, the spacer 40 may be provided with ribs to aid the user's grip when attaching and detaching the spacer 40.

FIG. 9 shows the use of a thermoplastic elastomer co-moulding 73 for forming the outlet tip of a nasal actuator. A rigid core 72 of polymeric plastics or metal which defines an outlet orifice 71 is overlaid by a layer 73 of thermoplastic elastomer of the type described above. Preferably, the thermoplastic elastomer extends over the entire surface contacted by the user's nose whilst leaving the internal surface 74 of the outlet orifice 71 free of thermoplastic elastomer. In this way the spray pattern which depends on the orifice geometry is not adversely affected. Advantageously, the thermoplastic elastomer coating 73 provides a comfortable, soft and hygienic contact surface for the user. In addition the material feels ‘warmer’ to the touch which has been found to be a desirably characteristic. The thermoplastic elastomer is formed as a co-moulding as described above.

The concept of the present invention may be applied to other types of dispensing apparatus such as ophthalmic dispensers, dermal applicators (transdermal or hypodermic), needleless injectors, aural dispensers and vaginal dispensers. Any of the thermoplastic elastomer components described with reference to the oral actuators of FIGS. 1 to 6b and nasal actuator of FIG. 8 may be incorporated in such dispensing apparatus as appropriate. For example a thermoplastic elastomer-coated tip may be provided on an aural dropper for use with ear drops or thermoplastic elastomer ribs may be provided on the housing of a needleless actuator.

1. A hand held dispensing apparatus comprising an outlet through which, in use, product is dispensed from a container of stored product locatable within, or communicating with, the apparatus wherein at least a portion of an exterior surface of the apparatus is formed from a thermoplastic elastomer.

2. A hand held dispensing apparatus as claimed in claim 1, wherein at least a portion of the outlet contactable by a user during use of the apparatus is formed from a thermoplastic elastomer.

3. A hand held dispensing apparatus as claimed in claim 1 or claim 2, wherein means are provided for sealing the outlet when the apparatus is not in use, said means being formed from a thermoplastic elastomer.

4. A hand held dispensing apparatus as claimed in any preceding claim, wherein at least one of the portions of thermoplastic elastomer on the exterior surface of the apparatus forms a non-slip surface to aid handling of the apparatus.

5. A hand held dispensing apparatus as claimed in any preceding claim wherein at least one of the portions of thermoplastic elastomer on the exterior surface of the apparatus has a raised profile.

6. A hand held dispensing apparatus as claimed in any preceding claim, wherein the thermoplastic elastomer portion is formed as a co-moulding with the remainder of the apparatus.

7. A hand held dispensing apparatus as claimed in claim 6 wherein at least one of the portions of thermoplastic elastomer on the exterior surface of the apparatus is keyed into a recess in the apparatus.

8. A hand held dispensing apparatus as claimed in any of claims 1 to 5 wherein at least one of the portions of thermoplastic elastomer on the exterior surface of the apparatus is coated on the apparatus.

9. A hand held dispensing apparatus as claimed in any preceding claim, wherein the apparatus is an actuator for containing a dispensing container operable to dispense an aerosol spray.

10. A hand held dispensing apparatus as claimed in claim 9, for use with a pressurised dispensing container.

11. A hand held dispensing apparatus as claimed in claim 9, for use with a dispensing apparatus using a non-liquified gas system.

12. A hand held dispensing apparatus as claimed in any of claims 1 to 8, wherein the apparatus is an inhalator operable to dispense a powdered medicament.

13. A hand held dispensing apparatus as claimed in any of claims 1 to 8, wherein the apparatus is a spacer.

14. A hand held dispensing apparatus as claimed in any of claims 1 to 8, wherein the apparatus is an ophthalmic dispenser.

15. A hand held dispensing apparatus as claimed in any of claims 1 to 8, wherein the apparatus is a dermal applicator.

16. A hand held dispensing apparatus as claimed in any of claims 1 to 8, wherein the apparatus is a needleless injector.

17. A hand held dispensing apparatus as claimed in any of claims 1 to 8, wherein the apparatus is an aural dispenser.

18. A hand held dispensing apparatus as claimed in any of claims 1 to 8, wherein the apparatus is a vaginal dispenser.

19. A hand held dispensing apparatus as claimed in any of claims 1 to 8, wherein the apparatus is a nasal actuator.

20. A hand held dispensing apparatus as claimed in claim 19 wherein the outlet of the nasal actuator comprises a rigid internal core defining an outlet passage and an exterior lining formed from a thermoplastic elastomer.

21. A hand held dispensing apparatus as claimed in any preceding claim further comprising a releasable closure for sealing the outlet wherein the closure is formed from a thermoplastic elastomer.

22. A hand held dispensing apparatus as claimed in any preceding claim, wherein the thermoplastic elastomer is one of Santoprene, Pebax, Vitaprene or Hytrel or the like.

23. Apparatus for dispensing a medicament, comprising a housing defining an outlet through which, in use, medicament is dispensed, a removable cap engageable with the outlet to close the outlet, and a strap connecting the cap to the housing whereby the cap is still attached to the housing when disengaged from the outlet, wherein the strap is formed from a thermoplastic elastomer having sufficient elasticity to accommodate the engagement and disengagement of the cap with the outlet.

24. Apparatus as claimed in claim 23 wherein the length of the strap is such that the strap lies in close proximity with the housing when the cap is engaged with the outlet.

25. Apparatus as claimed in claim 23 or claim 24 wherein the cap is formed from a thermoplastic elastomer.

26. Apparatus as claimed in claim 25 wherein the cap and strap are formed as a unitary body.

27. Apparatus as claimed in any of claims 23 to 26 wherein a pad of thermoplastic elastomer material is formed at one end of the strap, the pad being fixedly attached to the housing to form a non-slip surface.

28. Apparatus as claimed in claim 7 wherein the pad, strap and cap are formed as a unitary body.

29. Apparatus as claimed in claim 28 wherein one of the housing or the unitary body comprising the pad, strap and cap is formed in a first moulding step and the other of the housing or the unitary body comprising the pad, strap and cap is formed in a second moulding step.

30. Apparatus as claimed in any of claims 23 to 29 wherein one or more portions of the housing are coated with thermoplastic elastomer to form a non-slip surface.

31. Apparatus as claimed in any of claims 23 to 30 wherein movement of the cap into and out of engagement with the outlet is accompanied by co-axial relative movement of the cap and the outlet.

32. Apparatus as claimed in any of claims 23 to 31 wherein the apparatus comprises a pressurised dispensing container operable to dispense an aerosol spray.

33. Apparatus as claimed in any of claims 23 to 31 wherein the apparatus comprises an inhalator operable to dispense a powdered medicament.

34. Apparatus as claimed in any of claims 23 to 31 wherein the apparatus comprises a nasal actuator.

35. Apparatus as claimed in any of claims 23 to 31 wherein the apparatus is a spacer.
